(19) United States
(12) Patent Application Publication
(10) Pub. No.: US 2005/0061314 A1
(43) Pub. Date: Mar. 24, 2005

(54) Metered dose inhaler

(75) Inventors: Rebecca Jayne Davies, Chippenham (GB); David Ganderton, Chippenham (GB); David Lewis, Chippenham (GB); Brian Meakin, Chippenham (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Vactura Limited

(21) Appl. No.: 10/499,288

(22) PCT Filed: Dec. 23, 2002
(86) PCT No.: PCT/GB02/05903

(30) Foreign Application Priority Data
Dec. 21, 2001 (EP) 01130521.6
Dec. 19, 2002 (WO) PCT/EP02/14588

(51) Int. Cl. A61M 11/00

(52) U.S. Cl. 128/200.23; 128/200.14

(57) ABSTRACT

A medicinal aerosol product comprising a pressurized metered dose inhaler, including a canister (1) equipped with a metering valve and containing a medicinal aerosol solution formulation, and an actuator (2) comprising a nozzle block (14) defining an actuator orifice (6) leading to an expansion chamber, wherein the formulation includes a cannabinoid, a hydrofluorocarbon propellant and an optional amount of an alcohol co-solvent, and the actuator orifice (6) has a diameter of about 0.30 mm or less, and/or is laser drilled.

### DESCRIPTION

The present invention relates to pressurized metered dose inhaler (pMDI) devices, actuators used in such devices and to medicinal aerosol solution formulation products comprising such actuators. In particular, the present invention relates to medicinal aerosol products that comprise cannabinoid solution formulations that include hydrofluoroalkane (HFA) propellants, contained within pMDI devices. The pMDI actuators employed in medicinal aerosol products in accordance with the invention have delivery orifices of a specified size and type, that are preferably laser drilled. The cannabinoid solution formulations can include a co-solvent.

In certain preferred embodiments, medicinal aerosol products in accordance with the invention include formulations with a high co-solvent to active ingredient (cannabinoid) ratio.

There are over 70 different compounds that have been identified in extracts derived from cannabis plants. They are all substituted monoterpenes and are collectively referred to as cannabinoids. The major cannabinoids include the following compounds and their derivatives: Δ⁹-tetrahydrocannabinol (Δ⁹-THC), Δ⁸-tetrahydrocannabinol (Δ⁸-THC), cannabidiol (CBD), cannabinol (CBN), cannabigerol (CBG), cannabichromene (CBC), cannabicyclol (CBL), cannabielsoin (CBE), cannabinodiol (CBND), cannabitriol (CBO). The main source of these compounds is the Cannabis sativa plant, although some cannabinoids have been synthesised and certain derivatives are synthesised from naturally derived precursors.

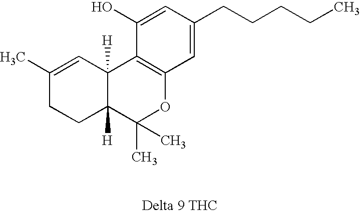

Derivatives of the specific compounds listed above include compounds with a carboxyl group bound to the phenolic ring, a methyl, propyl or butyl side chain in place of the pentyl side chain in the above structure, and/or a methoxy group in place of one or more of the basic compound's hydroxyl moieties. Most natural cannabinoids have at least two chiral centres at carbons 10a and 6a. Other cannabinoids, that are present in cannabis in minor amounts, include dehydrocannabifuran (DCBF), cannabifuran (CBF), cannabicitran (CBI), cannabichromanon (CBCN), a dimetic cannabinoid formed by esterification of cannabidiolic acid with tetrahydrocannabitriol and cannabinerolic acid (the trans isomer of CBG). The most important natural cannabinoid is the psychoactive Δ⁹-THC, which was isolated in 1964. The other important pharmacologically active cannabinoids are CBN, CBD, CBC and CBG. CBN potentates the effect of THC in man and antagonises cataleptic effect of THC in mice. CBD, which combats anxiogenic effect of THC, increases THC levels in the mouse brain. The THC:CBD ratio affects pain control and also blocks cytochrome 450-3A11 and the production of 11-hydroxy THC. CBC is a sedative and is synergistic with benzodiazepines and barbiturates but it is not cannabimetic, although it does lower LD₅₀ of THC in mice, and CBG is a more potent GABA inhibitor than THC, but is less potent in this respect than seratonin. Recently developed synthetic cannabinoids include Dexanabinol, which is a optical isomer of a derivative of THC, and CT-3, which is a synthetic derivative of carboxylic tetrahydrocannabinol (THC-7C) and has increased anti-inflammatory and analgesic properties and reduced psychotropic activity compared to THC. Synthetically produced ‘Dronabinol’ is identical with the natural RR-trans-Δ⁹-THC isomer. For the purposes of this specifcation, all such compounds and their derivatives should be understood as falling within the ambit of the term “cannabinoids”. The term should also be understood as encompassing extracts, which contain any of these compounds alone or in admixture with others, derived from a natural source such as the cannabis plant.

The preferred cannabinoids used in formulations in accordance with the invention include Δ⁹-THC and its salts and esters. Esters of the type described in WO 00/29402, U.S. Pat. No. 4,933,368 and U.S. Pat. No. 5,389,375 are particularly preferred, especially the Δ⁹-THC hemisuccinate described in WO 00/29402. This latter compound can be prepared by the method described in WO 00/29402.

The cannabis plant and products derived from it have been used for medicinal purposes for thousands of years. In more recent history, it has been found that smoking marijuana can provide relief from the nausea and vomiting associated with cancer chemotherapy and the spasticity caused by multiple sclerosis. Δ⁹-THC has been approved, in an oral dosage form (marketed as MARINOL®), by the US Food and Drug Administration (FDA) for the control of nausea and vomiting associated with chemotherapy and for appetite stimulation in patients suffering from AIDS wasting syndrome. Studies reported in the literature have also demonstrated that cannabinoids, particularly, Δ⁹-THC, have the potential to be therapeutically useful in the treatment of a number of conditions, including glaucoma, migraine headaches, spasticity in multiple sclerosis and as a result of spinal injury, muscle spasticity, pain, anorexia associated with cancer chemotherapy, epilepsy, mood disorders and asthma (as a bronchodilator).

The possibility of delivering cannabinoids by inhalation from a pMDI has been proposed by several different authors over the past 25-30 years. For example, in Williams et al. Thorax (1976), 31, 720, the authors reported that they had used pMDIs charged with a formulation consisting of a CFC propellant, Δ⁹-THC, and ethanol as a co-solvent, to elicit a bronchodilatory effect in asthmatic patients. In the same year, Olsen et al. proposed an alternative pMDI formulation for Δ⁹-THC that included, a CFC propellant, Δ⁹-THC and ethanol, plus a small quantity of sorbitan trioleate intended to detacify the Δ⁹-THC and to thereby improve the transport of the latter into the lungs. See Olsen et al. J. Pharm. Pharmac., 1976, 28, 86. More recently, formulations consisting of a hydrofluoroalkane (HFA), Δ⁹-THC and an optional quantity of ethanol and were proposed in WO 00/24362.

The pharmaceutical solution formulations in hydrofluoroalkanes used in the present invention may be filled into canisters suitable for delivering pharmaceutical aerosol formulations. Canisters generally comprise a container capable of withstanding the vapour pressure of the HFA propellant, such as plastic or plastic-coated glass bottle or preferably a metal can, for example a stainless steel can or aluminium can which is preferably anodised, organic coated, such as lacquer-coated and/or plastic coated (see WO00/30608), which container is closed with a metering valve. The metering valves comprising a metering chamber are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber are especially suitable. Suitable valves are commercially available from manufacturers well known in the aerosol industry, for example, from Valois, France (e.g. DF10, DF30, DF31, DF60), Bespak plc UK (e.g. BK300, BK356, BK357) and 3M-Neotechnic Ltd. UK (e.g. Spraymiser™).

Valve seals, especially the gasket seal, and also the seals around the metering chamber, will preferably be manufactured from a material which is inert to and resists extraction into the contents of the formulation, especially when the contents include ethanol.

Valve materials, especially the material of manufacture of the metering chamber, will is preferably be manufactured of a material which is inert to and resists distortion by contents of the formulation, especially when the contents include ethanol. Particularly suitable materials for use in manufacture of the metering chamber include polyesters eg polybutylenetetephthalate (PBI) and acetals, especially PBT.

A valve stem extends from the metering valve and acts as a conduit to pass the metered dose into a nozzle block situated in the actuator body, in which the valve stem is seated.

Materials of manufacture of the metering chamber and/or the valve stem may desirably be fluorinated, partially fluorinated or impregnated with fluorine containing substances in order to resist drug deposition.

Each filled canister is conveniently fitted into a suitable channelling device prior to use to form a metered dose inhaler for administration of the medicament into the lungs or nasal cavity of a patient. Suitable channelling devices comprise, for example a valve actuator and cylindrical or cone-like passage through which medicament may be delivered from the filled canister via the metering valve to the nose or mouth of a patient e.g. a mouthpiece actuator.

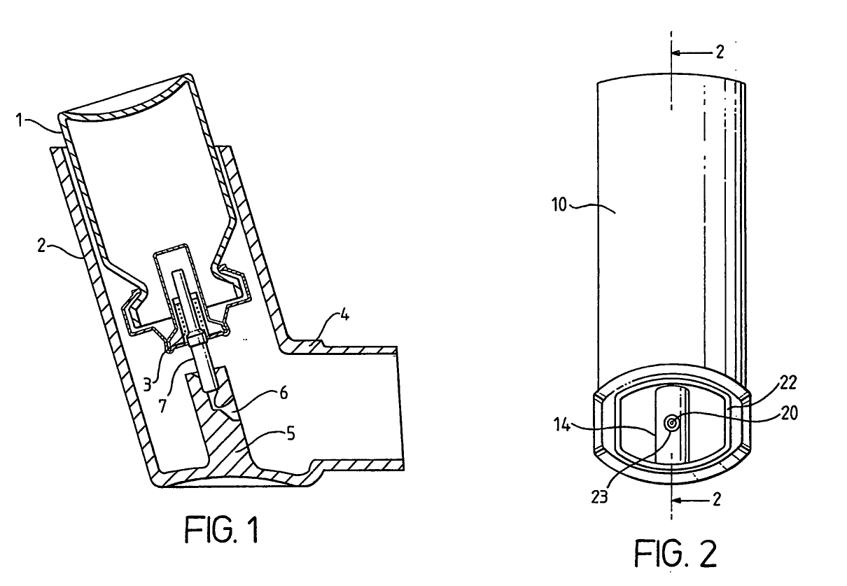

In a typical arrangement (see FIG. 1), the valve stem 7 is seated in a nozzle block which comprises an actuator insert 5, which comprises an actuator orifice 6 leading to an expansion chamber. Conventional pressurized metered dose inhaler actuators have variable actuator orifice diameters from 0.25 to 0.42 mm and a length from 0.30 to 1.7 mm. In other types of actuators the lengths can vary.

International Patent Application WO01/19342 discloses actuator orifice diameters in the range of 0.15 to 0.45 mm, particularly 0.2 to 0.45 mm. According to this prior art reference it is advantageous to use a small diameter e.g. 0.25 mm or less, particularly 0.22 mm since this tends to result in a higher FPM (fine particle mass) and lower throat deposition. Moreover it is stated that 0.15 mm is also particularly suitable. However, this prior art reference does not disclose how to obtain actuator orifices of less than 0.2 mm. The examples only relate to pMDIs having actuator orifices of 0.22 mm, 0.33 mm and 0.50 mm. Thus, although referring in general to small actuator orifice diameters of less than 0.2 mm, the prior art does not provide a solution how to obtain such small orifices with a high precision, i.e. with tightly controlled tolerances.

WO 01/58508 discloses an actuator for a metered dose inhaler containing a liquefied propellant and a medicament. The actuator comprises a nozzle block having a fluid flow path extending therethrough, the fluid flow path defined by an internal chamber having an inlet and an outlet; the outlet being defined in a portion of said nozzle block and comprising an exit channel extending therethrough. The exit channel has a narrow portion wherein the diameter of the channel is 0.3 mm or less, the narrow portion being 0.5 mm or less in length; and the narrow portion optionally including a constriction having a diameter of less than 0.3 mm. According to WO 01/58508, the increased degree of material deposition typically encountered with the use of nozzle orifices having a diameter of 0.3 mm or less may be reduced to a level at or below that experienced with larger diameter nozzles while still producing the high fine particle fractions achievable through using small diameter orifice nozzles (0.3 mm or less). This is accomplished by limiting the length of the portion of the nozzle channel which is 0.3 mm or less in diameter to 0.5 mm or less in length.

WO 99/55600 discloses a medicinal aerosol product having a blockage resistant metered-dose valve with a metal valve stem, particularly for use with CFC-free solution formulations using hydrogen containing propellants, such as 134a and/or 227, and ethanol. Moreover, a metered dose inhaler comprising an actuator and an aerosol product is disclosed. The actuator comprises a nozzle block and a mouth piece, the nozzle block defining an aperture for accommodating the end of the valve stem and an orifice in communication with the aperture directed towards the mouth piece, the orifice having a diameter of less than 0.4 mm, preferably about 0.3 mm.

There is no suggestion in any of these documents that a cannabinoid formulation could be delivered effectively from a pMDI with anything other than a conventionally dimensioned delivery orifice.

Metered dose inhalers are designed to deliver a fixed unit dosage of medicament per actuation shot or “puff”, for example in the range of 25 to 250 μg medicament per puff, depending on the metering chamber volume used.

In the accompanying drawings,

FIG. 1 shows a conventional pressurized metered dose inhaler comprising a canister 1, an actuator 2, a metering valve 3 with a valve stem 7, an oral tube 4, and a nozzle block comprising an actuator insert 5 and an actuator orifice 6.

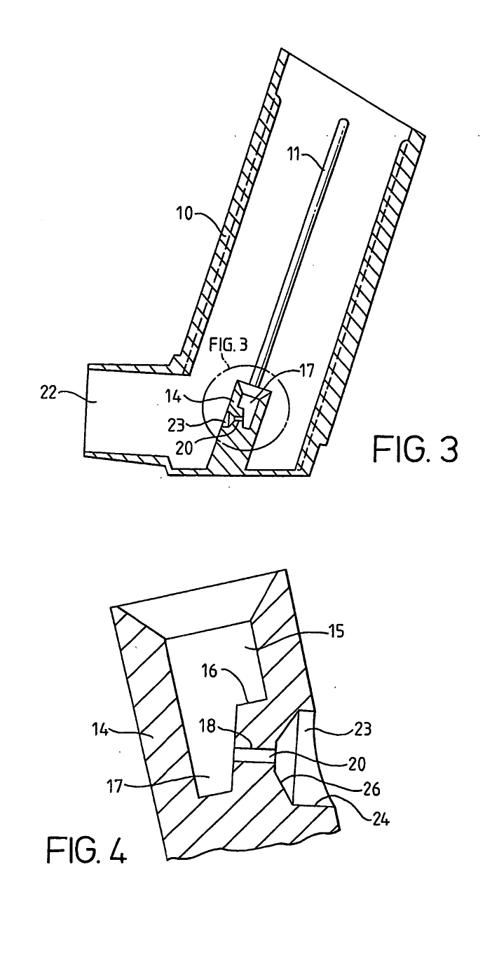

FIGS. 2, 3 and 4 show a conventional actuator nozzle block. FIG. 3 is a section on line 2-2 of FIG. 2, and FIG. 4 is an enlarged reversed view of the circled part of FIG. 3.

Referring to the figures, a conventional pressurized metered dose inhaler consists of a body portion 10 of an actuator into which a pressurized canister 1 containing a medicinal aerosol solution formulation may be inserted, and located by means of ribs 11.

A nozzle block 14 of the body portion 10 has a bore 15 which receives the valve stem 7 of the canister 1. The end of the stem beares on a step 16 within the base so that compressing the body portion 10 and canister 1 together opens the valve 3 and causes the discharge under pressure of a single measured quantity of the drug in its carrier medium.

The dose passes down a passage 17 in the nozzle block 14, through a conduit 18 (with an actuator orifice length), e.g. a parallel-bore conduit to a discharge nozzle 20 (with an actuator orifice diameter), and thence through a mouthpiece 22 of the body portion 10 of the actuator.

The shape and direction of the discharge plume and the dispersion of the droplets or particles therein are critical to effective administration of a controlled dose to the patient.

Conventionally the discharge nozzle 20 is positioned in a cylindrical recess 23 in the nozzle block 14 having a parallel sided portion 24 and a frusto-conical base 26. In order for the patient to insert the mouthpiece at the correct orientation for discharge of the spray whilst at the same time holding the body portion 10 of the actuator and the canister 1 at a convenient angle, the axis of the mouthpiece 22 is inclined at an obtuse angle of about 105 degrees to that of the body portion 10 of the actuator and nozzle block 14. Because of this geometry, the conical recess is not perpendicular to the surface of the nozzle block 14, resulting in the parallel sided portion 24 being shorter on one side than the other.

The dimension of the discharge nozzle 20 (actuator orifice diameter) and the recess 23 are such that the discharge plume dose not impinge directly upon the sides of the recess 23.

A problem with known inhaler spray nozzles is that of adequately matching the dimensions of the conduit 18 (actuator orifice length) and nozzle 20 (actuator orifice diameter) to the particular drug formulation and carrier-propellant. Different drugs have different flow and dispersion characteristics (particularly as between suspensions wherein drug particles are dispersed in the formulation and solutions wherein the drug is completely dissolved in the formulation) and it is often difficult to achieve the optimum balance between the plume shape, total dose volume and plume duration.

It has been disclosed (See Leawis D. A. et al., Respiratog Drug Delivery VI, 363-364, 1998) that, when using commercially available actuators for delivering solution formulations of aerosol pressurized with HFA, a reduction in the delivery orifice diameter, or cross-sectional area, induces an increase in the fine particle dose (FPD) of the aerosol produced.

The FPD, which provides a direct measurement of the aerosol particles considered suitable for deposition and retention in the respiratory tract, is calculated as the mass of the particles deposited from stage 3 to the filter (particles with an aerodynamic diameter less than 4.7 μm) in an Andersen Cascade Impactor.

The aerodynamic particle size distribution of an aerosol formulation is characterised using a Multistage Cascade Impactor according to the procedure described in European Pharmacopoeia 2nd edition, 1995, part V.5.9.1, pages 15-17. Generally an Andersen Cascade Impactor (ACI) is utilised. Deposition of the drug on each ACI plate is determined by high performance liquid chromatography (HPLC). Mean metered dose is calculated from the cumulative deposition in the actuator and ACI stages; mean delivered dose is calculated from the cumulative deposition in the ACI. Mean respirable dose (fine particle dose, i.e. FPD) which provides a direct measurement of the aerosol particles is considered suitable for deposition and retention in the respiratory tract, is obtained from the deposition on Stage 3 (S3) to filter (AF) corresponding to particles with an aerodynamic diameter ≦4.7 μm. Smaller particles, with an aerodynamic diameter ≦1.1 μm correspond to the fraction obtained from the deposition on Stage 6 to filter.

The FPD can also be expressed as a percentage of the ex-valve dose or recovered dose (i.e. Fine Particle Fraction: FPF_{≦}4.7 μm or FPF_{≦}1.1 μm). Shot weights are measured by weighing each canister before and after the actuation.

Although those skilled in the art would expect solution formulations comprising Δ⁹-THC, an HFA and ethanol, such as those taught in WO 00/24362, to provide acceptable results when used with pMDI devices with conventional delivery orifice dimensions, they would not have expected such formulations to provide good results with devices that have smaller diameter delivery orifices. Instead, they would have expected the tacky nature of the Δ⁹-THC, or any other cannabinoid, to cause such smaller orifices to clog excessively unless a detacifier, of the nature proposed in Qisen et al. J. Pharm. Pharmac., 1976, 28, 86, were to be included in the formulation.

HFA solution formulations usually contain a co-solvent, generally an alcohol and usually ethanol to dissolve the active ingredient in the propellant. Depending on the concentration and solubility characteristics of the active ingredient, the concentration of solubilisation agent (e.g. ethanol) can increase. Larger amounts of ethanol increase the velocity of the aerosol droplets leaving the actuator orifice. The high velocity droplets extensively deposit into the oropharyngeal tract to the detriment of the dose which penetrates in the lower airways (i.e. respirable fraction or fine particle fraction (FPF)).

An object of the present invention is to provide a medicinal aerosol product, comprising a solution formulation of a cannabinoid and a hydrofluoroalkane (HFA) contained within a pressurized metered dose inhaler (PMDI) device, with enhanced output characteristics. Another object of the invention is to provide such a product that efficiently atomises such formulations, particularly those that contain higher levels of a co-solvent, for example ethanol, so as to provide, for example, a fine particle is fraction (i.e. particles with a diameter smaller than 4.7 μm) of at least 50%, preferably at least 60% and more preferably at least 70% (and in some cases at least 80%) and an enhanced balance of the plume shape, total dose volume and the plume duration.

According to the present invention, there is provided a medicinal aerosol product comprising a pressurized metered dose inhaler, including a canister equipped with a metering valve and containing a medicinal aerosol solution formulation, and an actuator comprising a nozzle block defining an actuator orifice leading to an expansion chamber, wherein the formulation includes a cannabinoid, a hydrofluorocarbon propellant and an optional amount of an alcohol co-solvent, and the actuator orifice is laser drilled and/or has a diameter of about 0.30, 0.25, 0.021, 0.20 or 0.18 mm or less.

Surprisingly, even when a detacifier of the aforementioned nature is not present in the formulation, the actuator orifice of a product in accordance with the invention does not become unacceptably clogged with use. Thus, products in accordance with the present invention can enjoy all of the aforementioned advantages associated with the use of a small actuator orifice cross-section, or diameter, and yet can be based upon a simple formulation with a minimum number of ingredients.

In certain preferred embodiments of the invention, the formulation is substantially free of added surface active agent, or detacifier, such as sorbitan trioleate.

Preferably, the actuator orifice has a diameter of about 0.10-0.30 mm, and more preferably in the range of about 0.10-0.20 mm. In preferred embodiments, the actuator orifice remains within the preferred diameter range over a length of at least about 0.30 mm, and preferably up to about 1.7 mm, and more preferably within a length of about 0.50-1.0 mm.

In embodiments, the actuator orifice can have a diameter below about 0.20 mm over the entire actuator orifice length. Preferably its diameter is in the range from 0.10 to 0.20 mm, more preferably 0.11 to 0.18 mm and in particular from 0.12 to 0.18 mm over the entire actuator orifice length, wherein diameters of 0.12 mm, 0.14 mm, 0.16 mm and 0.18 mm are particularly preferred. The orifice diameter can be different at the inlet and at the outlet of the actuator orifice, however, it should be in the given range over the entire actuator orifice length. Preferred orifice diameter combinations inlet/outlet (mm) are 0.12/0.18, 0.18/0.12, 0.14/0.18, 0.18/0.14, 0.16/0.18, 0.18/0.16, 0.12/0.16, 0.16/0.12, 0.14/0.16 and 0.16/0.14.

Small actuator orifice diameters can be obtained by using a laser to drill the actuator orifices. The advantages of using a laser to drill the actuator orifices include, very high precision down to a few microns, smooth interior bore, tightly controlled taper and dimensional tolerances, entry angle holes down to 10 degrees and minimal heat damage. Thus, the present invention provides for the first time an alternative to existing moulding techniques and provides pMDI actuators with very small actuator orifice diameters with tightly controlled tolerances which is necessary to be able to provide tightly controlled reproducability of the unit dosage of medicament per actuation.

In addition to the actuator orifice diameter, the actuator orifice length is an important feature according to the present invention. Preferably, the actuator orifice has a length in the range from 0.50 mm to 1.00 mm, in particular from 0.60 mm to 0.80 mm.

For example a copper vapour laser (CVL) (Oxford Lasers ltd.) can be used to produce actuators with tightly controlled tolerances on orifice diameter and length.

The dimensions of the actuator orifices are checked using a Mitntoyo TM WF20X microscope and Dolan-Jenner Fiberlite.

Combinations of actuator orifice diameter and length in accordance with the invention provide actuators with improved actuator blockage/device clogging characteristics with die cannabinol/hydrofluoroalkane solution formulation used in the inventive products, especially those that have a relatively high ethanol content and an optional quantity of a low volatility component such as glycerol.

Actuator orifice length of the nozzle blocks of the present invention refers to the distance between the external face (outlet) and the internal surface (inlet) which due to the design of the nozzle blocks are parallel.

In preferred embodiments, the medicinal aerosol products in accordance with the present invention contain a cannabinoid, a hydrofluorocarbon propellant such as HFA 134a, HFA 227 or a mixtures thereof, ethanol as a co-solvent and, optionally, a low volatility component, such as glycerol, propylene glycol, polyethylene glycol and isopropylmyristate. The ethanol is preferably present in an amount of at least about 2, 3, 4, or 5% and preferably up to about 10, 12, 15 or 20% by weight of the solution formulation. The formulation preferably comprises between about 0.10, 0.12, 0.14, 0.15, 0.16, 0.17 and 1.7, 1.75, 1.8, 2.0, 2.5 or 3% cannabinoid by weight.

As previously noted, the cannnabinoid is preferably Δ⁹-THC, a salt or ester and more preferably Δ⁹-THC hemisuccinate. The formulation can include a mixture of cannabinoids and the total amount of cannabinoids present in the formulation, preferably, lies within one of the aforementioned preferred cannabinoid concentration ranges. The cannabionoids can be in the form of an extract derived from the cannabis plant.

The low volatility component is preferably glycerol and is preferably employed in an amount of up to about 0.4, 0.3, 0.2 or 0.1% by weight of the solution formulation and can be used to increase the MMAD of the atomised particles if required.

The concentration of cannabinoid is selected to provide a dose of between about 25, 50, 75 or 100 μg to about 1000, 1100, or 1400 μg per valve actuation. In order to provide such a dose, the chamber in the metering valve, preferably, has a volume of between 25 and 100 μl; the most preferred metering valve chamber volumes are 50 and 63 μl.

The medicinal aerosol products of the present invention are able to produce an aerosolised medicament showing a fine-particle fraction of at least 50, 60, or 70% and an optimum balance of the plume shape, total dose volume and the plume duration despite the fact that they can include relatively high concentrations of co-solvent (ethanol). Moreover, blockage and clogging problems due to materials depositions are avoided. The use of these kinds of solution formulations results in particles with a MMAD (Mass Median Aerodynamic Diameter) that is preferably ≦2.5 or 2.0 μm. Thus, the present invention provides a medicinal aerosol solution for a medicinal aerosol solution formulation product comprising actuators with an extremely efficient atomisation in combination with solution formulations consisting substantially of a cannabinol, ethanol and a hydrofluorocarbon as propellant. If a further additive is present in the solution formulation, it is preferably only present in such an amount that it does not have any detrimental influence on the MMAD of the atomised particles.

The actuator orifice can be of circular or other cross-section, but it is preferably circular in cross-section. However, when the cross-section of the orifice is other than circular, it should have a cross-sectional area equal to that of a circular orifice with a diameter lying within one of the above defined preferred ranges.

In one embodiment of the invention, the nozzle structure is manufactured as a separate actuator insert piece which is fitted into the nozzle block 14. Alternatively or in addition, the nozzle block may be a separate component fitted into the body portion 10.

Preferably, the actuator insert pieces are constructed of aluminium or stainless steel, as using a CVL to micro drill plastic results in to much heat damage. However, according to one embodiment of the invention it is possible to laser drill into plastics without heat damage, by frequency doubling the visible output of the CVL. This generates three ultra-violet wave lengths, e.g. 255 nm, 271 nm and 289 nm. With these ultra-violet wave lengths plastics can be drilled to high precision without heat damage.

Any kind of actuator inserts known in the art, or of nozzle structures known in the art (e.g. as described in GB-A-2276101 and WO99/12596) can be provided with laser drilled orifices. Preferably, the actuator inserts or nozzle structures are made of aluminium or stainless steel.

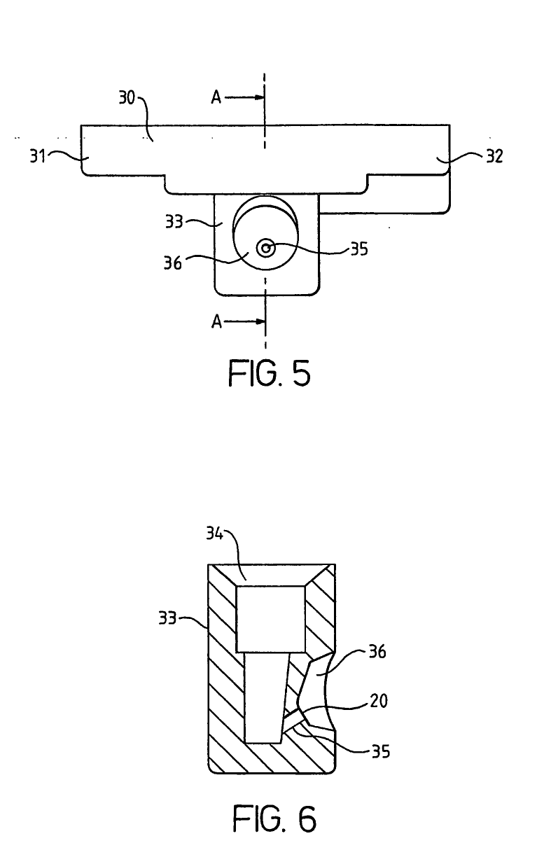

In one embodiment of the present invention an aluminium nozzle block known in the art as the “Chiesi Jet piece” is provided with a laser drilled orifice. FIGS. 5 and 6 show the dimensions of the “Chiesi Jet piece” used in the examples of the present invention. FIG. 5 is a front view of the T shaped nozzle block. FIG. 6 is a section view of the nozzle block along lines A-A of FIG. 5. The “Chiesi Jet piece” is a separate component fitted into the body portion 10. For a detailed description reference is made to international patent application WO99/12596.

The nozzle block (30) is shaped as a T, consisting of an upper bar composed by two fins (31, 32) to be housed and retained in two seats provided in the two shells forming the device and of a vertical stem (33) shorter than the horizontal upper bar.

The vertical stem (33) comprises a socket (34) provided with a seat to house a hollow stem of a pressurized can.

In the thickness of the stem (33) is bored a conduit (35) that connects the socket (34) with the mouth piece (22) of the device through the orifice (20) positioned in a recess (36).

### EXAMPLES

### Example 1

The “Chiesi Jet piece” was used as a model for the aluminium nozzle block in this example. Once drilled the aluminium nozzle block was housed in a modified Bespak 630 series actuator. Test pieces were also constructed and used to check the orifice entrance (inlet) and exit (outlet) diameters. Adjusting the laser power and focus controls the degree to which the walls of the orifice converge or diverge along its length. The dimensions of all actuator orifices were checked using a Mitntoyo TM WF20X microscope and Dolan-Jenner Fibetlite.

Table 1 shows the dimensions of a range of actuator orifice diameters from 0.10 min to 0.18 mm, with a 0.60 mm orifice length (n=2). The various shaped orifices that can be produced are-slot, cross-clover leaf and peanut. The dimensions of the peanut are shown in table 2. Multiple holed actuator orifices were also produced. The dimensions of the multiple holed orifices are included in table 2.

TABLE 1
Measured diameters of the milled actuator inserts with 0.60 mm orifice length.

| | | | | |
|---|---|---|---|---|
| Target diameter(mm) | 0.14 | 0.12 | 0.10 | 0.18 |
| OrificeLength (mm) | 0.60 | 0.60 | 0.60 | 0.60 |
| Diameter (mm) piece 1 | 0.135 ± 0.004 | 0.114 ± 0.002 | 0.092 ± 0.003 | 0.177 ± 0.006 |
| Diameter (mm) piece 2 | 0.138 ± 0.008 | 0.108 ± 0.007 | 0.087 ± 0.003 | 0.181 ± 0.004 |

TABLE 2
Measured diameters of milled actuator inserts with either
shaped or multiple holed orifices (* holes are 0.5 mm apart).

| | | | |
|---|---|---|---|
| Area Cf. (mm) | 0.10 | 0.12 | 0.12 |
| Orifice Shape | Peanut | 2 hole* | 4 hole* |
| x (mm) | 0.068 ± 0.003 | 0.083 ± 0.006 | 0.061 ± 0.005 |
| y (mm) | 0.116 ± 0.001 | | |

x indicates the extension in the horizontal direction;
y indicates the extension in the vertical direction;

Table 1 clearly shows the high precision that can be achieved with laser drilling into aluminium, as the measured orifice diameters closely match the target diameters. In table 2, the peanut cross-sectioned orifice has an area comparable to a 0.10 mm circular cross-sectioned actuator and was produced with two laser drillings.

### Exampl3 2

The experiments of example 2 consisted of discharging Δ⁹-THC (THC)/ethanol/glycerol/HFA 134a and Δ⁹-THC hemisuccinate (THC-HS)/ethanol/glycerol/HFA 134a formulations through actuator inserts housed in a modified Bespak actuator (630 series) into an Andersen Cascade Impactor operated at 28.3 L min⁻¹. Two product strengths, 100 μg/dose and 1000 μg/dose, were used in combination with 5% or 10% w/w ethanol, and 0.1% or 0.3% glycerol. Experiments were conducted using 0.14 and 0.16 mm diameter, 0.60 mm long, laser drilled actuator orifices in accordance with the invention in its preferred aspects and a third party supplied actuator with an orifice diameter of 0.22 mm. The quantities of drug deposited on the actuator, the throat and the stages of the impactor were measured. The delivered dose, the mass median aerodynamic diameter (MMAD), the geometric standard deviation (GSD), the fine particle dose ≦4.7 μm (FPD_{≦4.7}) and the fine particle dose ≦1.1 μm (FPD_{≦}1.1) were calculated from the results of these experiments and are set out in tables 3 and 4. The FPDs were also expressed as a % fraction of the ex-valve dose (FPF_{≦4.7}, FPF_{≦1.1}). Shot weight was measured by weighing the pMDI before and after discharge. These latter parameters are also set out in tables 3 and 4 along with other results of these experiments.

These figures show that products in accordance with the invention provide enhanced results, especially those products which include certain preferred features of the invention, such as smaller actuator orifice diameters and preferred cannabinoids.

| | |
|---|---|
| Actuator 0.14 mm | Actuator 0.14 mm |
| THC-HS, 5% Ethanol, | THC, 5% Ethanol, |
| 0.1% Glycerol, | 0.1% Glycerol, |
| HFA134a, 50 μl | HFA134a, 50 μl |

| | 100 μg | 1000 μg | 100 μg | 1000 μg |
|---|---|---|---|---|
| Recovered (μg) | 101.90 | 871.8 | 114.7 | 1109.1 |
| Delivered (μg) | 97.30 | 834.3 | 108.2 | 1044.5 |
| Actuator (μg) | 4.57 | 37.52 | 6.49 | 64.54 |
| Actuator (%) | 4.48 | 4.3 | 5.66 | 5.82 |
| Throat (μg) | 3.51 | 26.58 | 4.05 | 61.26 |
| Throat (%) | 3.44 | 3.05 | 3.53 | 5.52 |
| Stage 0-2 (μg) | 1.89 | 35.84 | 2.47 | 76.1 |
| Stage 0-2 (%) | 1.85 | 4.11 | 2.15 | 6.86 |
| FPD_{<4.7 μm (μg)} | 91.94 | 771.92 | 101.71 | 907.14 |
| FPF_{<4.7 (%)} | 90.23 | 88.54 | 88.67 | 81.79 |
| FPD_{<3.3 μm (μg)} | 89.66 | 676.16 | 98.02 | 735.5 |
| FPF_{<3.3 μm (%)} | 87.99 | 77.56 | 85.46 | 66.32 |
| FPD_{<1.1 μm (μg)} | 37.56 | 138.04 | 42.01 | 129.7 |
| FPF_{<1.1 μm (%)} | 36.86 | 15.83 | 36.63 | 11.69 |
| MMAD (μm) | 1.3 | 1.9 | 1.3 | 2.3 |
| GSD | 1.7 | 1.7 | 1.8 | 1.7 |
| Shot Weight(mg) ± SD | 64.7 ± 0.5 | 64.7 ± 0.6 | 62.4 ± 0.8 | 63.8 ± 0.4 |

| | THC-HS | THC | | THC-HS | THC |
|---|---|---|---|---|---|
| | 106.3 μg | 112.5 μg | | 968.3 μg | 1042.5 μg |
| Recovered (μg) | 101.90 | 114.7 | Recovered (μg) | 871.8 | 1109.1 |
| Delivered (μg) | 97.30 | 108.2 | Delivered (μg) | 834.3 | 1044.5 |
| Actuator (μg) | 4.57 | 6.49 | Actuator (μg) | 37.52 | 64.54 |
| Actuator (%) | 4.48 | 5.66 | Actuator (%) | 4.3 | 5.82 |
| Throat (μg) | 3.51 | 4.05 | Throat (μg) | 26.58 | 61.26 |
| Throat (%) | 3.44 | 3.53 | Throat (%) | 3.05 | 5.52 |
| Stage 0-2 (μg) | 1.89 | 2.47 | Stage 0-2 (μg) | 35.84 | 76.1 |
| Stage 0-2 (%) | 1.85 | 2.15 | Stage 0-2 (%) | 4.11 | 6.86 |
| FPD_{<4.7 μm (μg)} | 91.94 | 101.71 | FPD_{<4.7 μm (μg)} | 771.92 | 907.14 |
| FPF_{<4.7 (%)} | 90.23 | 88.67 | FPF_{<4.7 (%)} | 88.54 | 81.79 |
| FPD_{<3.3 μm (μg)} | 89.66 | 98.02 | FPD_{<3.3 μm (μg)} | 676.16 | 735.5 |
| FPF_{<3.3 μm (%)} | 87.99 | 85.46 | FPF_{<3.3 μm (%)} | 77.56 | 66.32 |
| FPD_{<1.1 μm (μg)} | 37.56 | 42.01 | FPD_{<1.1 μm (μg)} | 138.04 | 129.7 |
| FPF_{<1.1 μm (%)} | 36.86 | 36.63 | FPF_{<1.1 μm (%)} | 15.83 | 11.69 |
| MMAD (μm) | 1.3 | 1.3 | MMAD (μm) | 1.9 | 2.3 |
| GSD | 1.7 | 1.8 | GSD | 1.7 | 1.7 |
| Shot Weight(mg) ± SD | 64.7 ± 0.5 | 62.4 ± 0.8 | Shot Weight(mg) ± SD | 64.7 ± 0.6 | 63.8 ± 0.4 |

TABLE 4

| | | | | 0.22 mm |
|---|---|---|---|---|
| | 0.14 mm Aluminium | | 0.16 mm Al | Bespak |
| | insert (4b) | | insert (11A) | Actuator |
| Δ⁹ THC-HS 100 μg/ | 5% Et, | 10% Et, | 5% Et, | 5% Et, |
| 50 μl/134a | 0.1% Gly | 0.3% Gly | 0.1% Gly | 0.1% Gly |
| Recovered (μg) | 101.90 | 83.80 | 98.55 | 105.70 |
| Delivered (μg) | 97.30 | 79.50 | 93.40 | 98.00 |
| Actuator (μg) | 4.57 | 4.35 | 4.88 | 7.71 |
| Throat (μg) | 3.51 | 5.93 | 7.52 | 13.82 |
| Stage 0-2 (μg) | 1.89 | 2.60 | 2.28 | 2.12 |
| Stage 0-2 (%) | 1.85 | 3.10 | 2.31 | 2.01 |
| FPD < 4.7 μm (μg) | 91.94 | 70.93 | 83.56 | 82.02 |
| FPF < 4.7 (%) | 90.23 | 84.64 | 84.79 | 77.60 |
| FPD < 3.3 μm (μg) | 89.66 | 65.50 | 80.07 | 77.43 |
| FPF < 3.3 μm (%) | 87.99 | 78.16 | 81.25 | 73.25 |
| Dose < 1.1 μm (μg) | 37.56 | 14.21 | 28.19 | 24.30 |
| FPF < 1.1 μm (%) | 36.86 | 16.96 | 28.59 | 22.99 |
| MMAD (μm) | 1.3 | 1.7 | 1.4 | 1.5 |
| GSD | 1.7 | 1.7 | 1.8 | 1.7 |
| Shot Weight(mg) ± | 64.7 ± 0.5 | 60.7 ± 0.6 | 62.0 ± 0.6 | 62.6 ± 0.4 |
| SD | | | | |

1. A medicinal aerosol product comprising a pressurized metered dose inhaler including a canister, equipped with a metering valve and containing a medicinal aerosol solution formulation, and an actuator comprising a nozzle block defining an actuator orifice, wherein the formulation includes a cannabinoid, a hydrofluorocarbon propellant and an optional amount of an alcohol co-solvent, and the actuator orifice has a diameter of about 0.30 mm or less.

2. A medicinal aerosol product as claimed in claim 1, wherein the actuator orifice has a diameter of 0.25, 0.21, 0.20 or 0.18 mm or less.

3. A medicinal aerosol product as claimed in claim 1, wherein the formulation is substantially free of added surface active agent, or detacifier.

4. A medicinal aerosol product as claimed in claim 1, wherein the actuator orifice has a diameter of about 0.10-0.20 mm.

5. A medicinal aerosol product as claimed in claim 2, wherein the actuator orifice has a diameter of at least about 0.01 mm.

6. A medicinal aerosol product as claimed in claim 1, wherein the actuator orifice remains within the stated diameter range over a length of at least about 0.30 mm.

7. A medicinal aerosol product as claimed in claim 6, wherein the actuator orifice remains within the stated diameter range over a length of about 0.50-11.0 mm.

8. A medicinal aerosol product as claimed in claim 1, wherein the actuator orifice remains within the stated diameter range throughout its entire length and has a length of from about 0.50 mm to about 1.00 mm.

9. A medicinal aerosol product as claimed in claim 1, wherein the hydrofluorocarbon propellant is HFA 134a, HFA 227 or a mixtures thereof.

10. A medicinal aerosol product as claimed in claim 1, wherein the alcohol co-solvent is ethanol.

11. A medicinal aerosol product as claimed in claim 1, wherein the formulation further comprises a low volatility component.

12. A medicinal aerosol product as claimed in claim 11, wherein the low volatility component is glycerol, propylene glycol, polyethylene glycol or isopropylmyristate.

13. A medicinal aerosol product as claimed in claim 10, wherein the ethanol is present in an amount of at least about 2, 3, 4, or 5% by weight of the solution formulation.

14. A medicinal aerosol product as claimed in claim 10, wherein the ethanol is present in an amount of up to about 10, 12, 15 or 20% by weight of the solution formulation.

15. A medicinal aerosol product as claimed in claim 1, wherein the formulation comprises between about 0.10, 0.12, 0.14, 0.15, 0.16 or 0.17, and 1.7, 1.75, 1.8, 2.0, 2.5 or 3% cannabinoid by weight.

16. A medicinal aerosol product as claimed in claim 1, wherein the cannabinoid is Δ⁹-THC, a salt or ester thereof.

17. A medicinal aerosol product as claimed in claim 1, wherein the cannabinoid is Δ⁹-THC hemisuccinate.

18. A medicinal aerosol product as claimed in claim 1, wherein the formulation includes a mixture of cannabinoids.

19. A medicinal aerosol product as claimed in claim 18, wherein the cannabionoid mixture is in the form of an extract derived from a cannabis plant.

20. A medicinal aerosol product as claimed in claim 11, wherein the low volatility component is glycerol and is employed in an amount of up to about 0.4, 0.3, 0.2 or 0.1% by weight of the solution formulation.

21. A medicinal aerosol product as claimed in claim 1, wherein the actuator orifice is in the shape of a slot, cross, clover leaf or peanut.

22. A medicinal aerosol product as claimed in claim 1, wherein the nozzle block comprises two or a plurality of orifices.

23. A medicinal aerosol product as claimed in claim 1, wherein the nozzle block and/or the actuator insert piece is made of aluminium or stainless steel.

24. A medicinal aerosol product as claimed in claim 1, for use in medicine.

25. Use of a medicinal aerosol product as claimed in claim 1, for the manufacture of a medicament for the treatment of disease.

26. A medicinal aerosol product as claimed in claim 1, wherein said actuator orifice is laser drilled.
